(19) United States
(12) Patent Application Publication
(10) Pub. No.: US 2009/0275058 A1
(43) Pub. Date: Nov. 5, 2009

(54) METHOD FOR SPECIFIC DETECTION OF SALMONELLA SPP.

(75) Inventors: Laia Calvo, Girona (ES); Jesus Garcia-Gil, Girona (ES)

Correspondence Address: BERENBAUM WEINSHIENK PC, 370 17TH STREET, SUITE 4800, DENVER, CO, 80202, US

(73) Assignee: UNIVERSITAT DE GIRONA, Girona (ES)

(21) Appl. No.: 12/441,333

(22) PCT Filed: Sep. 12, 2007
(86) PCT No.: PCT/EP2007/059603

§ 371 Date: Mar. 13, 2009

(30) Foreign Application Priority Data
Sep. 14, 2006 (EP) 06120684.3

(51) Int. Cl. G01N 33/569

(52) U.S. Cl. 435/7.35

(57) ABSTRACT

A method for specific detection of the presence of Salmonella spp. in a sample that is suspected to contain Salmonella spp. and which further comprises one or more other microorganism(s).

### FIELD OF INVENTION

The present invention relates to a method for specific detection of the presence of Salmonella in a sample that is suspected to contain Salmonella, and which further comprises one or more other microorganism(s).

### BACKGROUND

Salmonella is a gram-negative, rod-shaped nonspore forming bacterium. The genus Salmonella is a member of the family Enterobacteriaceae, and encompasses two species: Salmonella bongori and Salmonella enterica. S. enterica includes six subspecies of clinical importance for humans causing million of cases of food borne disease in the world every year.

Up to five days are required for detecting Salmonella by means of traditional culture-based methods. Therefore, the need for new, quick and sensitive methods to detect Salmonella is a main concern in food safety.

Detection of Salmonella is nowadays being performed both on an alimentary sample and clinically.

Alimentary samples suspected to contain salmonella include for example egg, poultry, raw (under cooked) meat, raw seafood, milk, and dairy products, water, sauces and salad dressings, etc.

In clinical samples, Salmonella can be directly obtained from e.g. faeces of e.g a human.

Recently, the molecular detection by means of PCR-based techniques has become a common procedure for the rapid identification of Salmonella. Both conventional and modern real-time PCR protocols have been implemented, targeting a number of genes containing unique, signature sequences.

PCR-based methods described to date, target a number of phylogenetic and functional genes including oligonucleotides specifically targeting regions of the ribosomal operon such as the 16S subunit (Lin and Tsen (1996) J Appl Bacteriol 80, 659-666).

However, functional genes involved in virulence and infectivity are currently the markers of choice for most PCR procedures. The most widely used gene to date is invA (invasion A). The gene inv encodes for an essential component of the invasion-associated protein secretion apparatus, and is the first gene of the locus inv. This locus allows Salmonella spp. to enter epithelial cells causing an infection (Galán, et al, (1992) J Bacteriol, 174, 1338-4349). Other authors use genes as tyv, prt, viaB, flic-d or flic-a to detect and identify Salmonella enterica serovars Typhi and Paratyphy. These genes are O, H and Vi antigen genes (Hirose, et al, (2002) J Clin Microbiol 40, 633-636). Genes in the locus ttrRSBCA, which is required for tetrathionate respiration and located near the pathogenicity island 2 of Salmonella, are also used as a target to detect Salmonella in food by Real-time PCR (Malorny, et al, (2004) Appl. Environ. Microbiol. 70, 7046-7052).

However, all these genes present problems of non-specific amplifications as well as problems of inclusitivity (Cohen, et al, (1996) Appl. Environ. Microbiol. 62, 4303-4308).

### SUMMARY OF THE INVENTION

The problem to be solved by the present invention is to provide a method for specifically detecting Salmonella.

The solution is based on that the present inventors have identified that a specific Salmonella gene known under the term bipA (or typA) comprises sufficient specific sequences usable to specifically detect Salmonella in a sample, which further comprises one or more other microorganism(s) such as one or more other specie(s) than Salmonella.

The gene bipA (or typA) belongs to the “GTP-binding elongation family” of genes, category N. BipA (or typA) genes are known in different organisms such as e.g. E. coli and Bordetella spp. It is also known to be present in Salmonella (see below for further details). The induction of bipA (or typA) allows modulating a range of downstream processes including DNA metabolism and type III secretion. A ‘global regulatory’ gene such as bipA (or typA) is critical for cell growth and may be termed a “house-keeping” gene. It is known to the skilled person that such “house-keeping” genes are generally quite conserved within different species of a genus. However, as said above, surprisingly the bipA (or typA) gene of Salmonella as described herein comprises sufficient specific sequences usable to specifically distinguish Salmonella from other different species. See e.g. results 2.3 of working examples herein, where it is demonstrated that Salmonella can be specifically distinguished from a number of other relevant microorganisms. The results provided in the results 2.3 section are based on real-time PCR using genomic DNA and primers oriented towards a Salmonella bipA (or typA) gene as described herein.

Furthermore, functional genes like the ones listed in the background section above are normally subjected to strong variability, mainly because silent mutations in the third base of the codon. This means that in for example a 21-base pairs oligonucleotide, up to seven positions are in risk to be nonspecific, due to natural genetic variability of bacterial populations, which can compromise the specificity of the PCR system. For some reason, the gene bipA (or typA) as discussed herein, does not show this variability, making it an ideal target because of its highly conserved sequence.

The whole genome sequence of Salmonella enterica serovar Typhimurium LT2 is described in [McClelland, et al, (2001), Complete genome sequence of Salmonella enterica serovar Typhimurium LT2., Nature, 413, 852-856]. The genome sequence of Salmonella enterica serovar Typhi CT18 is described in [Parkhill, et al, (2001), Complete genome sequence of a multiple drug resistant Salmonella enterica serovar Typhi CT18. Nature, 413, 848-852]. The whole genome sequence of Salmonella enterica serovar Choleraesuis is described in Chiu, et al, (2005), The genome sequence of Salmonella enterica serovar Choleraesuis, a highly invasive and resistant zoonotic pathogen. Nucl. Ac. Res., 33, 1690-1698]. These complete genome sequences have the GenBank accession numbers AE006468, AL513382, and AE017220, respectively. The herein described bipA (or typA) gene of Salmonella is described in [Barker H C, Kinsella N, Jaspe A, Friedrich T, O'Connor CD (2000) Formate protects stationary-phase Escherichia coli and Salmonella cells from killing by a cationic antimicrobial peptide. Mol. Microbiol. 35: 1518-1529. The bipA (or typA) gene has the GenBank accession number: “STY276889 REGION: 12.845” and the DNA sequence is shown in SEQ ID NO 1. The corresponding amino acid sequence is herein termed BipA (or typA) GTPase and has the protein ID: CAC14270.1 and the amino acid sequence is shown in SEQ ID NO 2.

It is known to the skilled person that there may be some relatively minor sequence differences among the similar genes within different subspecies (or serovars) of specie of interest (here Salmonella). Based on common general knowledge and current available bioinformatics it is routine for the skilled person to identify such relatively minor sequence difference and determine if a gene of interest in specific subspecies is equivalent to a similar gene in another subspecies.

Accordingly, a first aspect of the invention relates to a method for specific detection of the presence of Salmonella in a sample that is suspected to contain Salmonella and which further comprises one or more other microorganism(s), characterized by that

(i): the sample is analyzed to identify for presence of a Salmonella bipA (or typA) gene; and
(ii) the amount of the Salmonella bipA (or typA) gene present in the sample is evaluated and if the sample comprises the Salmonella bipA (or typA) gene it is a proof for that Salmonella is present in the sample;
wherein the Salmonella bipA (or typA) gene is a bipA (or typA) gene consisting of: a bipA (or typA) gene comprising a DNA sequence which is at least 95% identical to the DNA sequence shown in positions 1-1824 of SEQ ID NO 1 (termed “bipA (or typA)”);
(a1) a bipA (or typA) gene that encodes a polypeptide which is at least 95% identical to the polypeptide shown in positions 1-607 of SEQ ID NO 2 (termed “BipA (or typA) GTPase”).

The term “sample that is suspected to contain Salmonella” relates to the objective of the method of the present invention, which is to analyze if the sample comprises Salmonella. Said in other words, if one is 100% sure that the sample comprises Salmonella or viable Salmonella there is no significant reason to analyze for the presence of it.

Obviously, the method may also involve detection of other e.g. relevant genes beside the bipa gene as described herein.

### DETAILED DESCRIPTION OF THE INVENTION

### Sample

The sample may e.g. be a clinical sample (preferably obtained from a human) or be a so-called alimentary sample. In clinical samples, Salmonella can be directly obtained from e.g. faeces of e.g a human such as a human patient with an intestinal disorder.

In a preferred embodiment the sample is an alimentary sample.

Preferably the “alimentary sample” is a sample obtained from a food or feed product or a food or feed precursor sample. A food or feed precursor sample is a sample which is subjected or be used in the preparation of a food or feed product. Preferably, the “alimentary sample” is a food product or food precursor sample intended to be used for human consumption.

Alimentary samples suspected to contain Salmonella include for example egg, water, poultry, raw (under cooked) meat, milk, seafood, raw vegetables, sausage, ice creams, etc.

As said above the sample further comprises one or more other microorganism(s).

Examples of other microorganisms include one or more microorganism(s) selected from the group consisting of other (not Salmonella) microorganism(s) of the Family Enterobacteriaceae.

Typical examples of such (not Salmonella) microorganism(s) of the Family Enterobacteriaceae include E. coli, Shigella, Enterobacter, Serratia, and Proteus.

The sample may also comprise microorganisms from other family than enterobacteriaceae. Examples of these include Micrococcus, Bacillus, Staphylococcus, Pseudomonas, Enterococcus, Arthrobacter and Listeria.

Preferably, the Salmonella to be detected as described herein is Salmonella typhimurium.

BipA (or typA) Gene

As explained above, the gene bipA (or typA) belongs to the “GTP-binding elongation family” family of genes, category N. BipA (or typA) gene is known in different organisms including Salmonella.

The term bipA (or typA) gene is widely known to the skilled person and based on his common general knowledge the skilled person can routinely determine whether or not a gene of interest is a bipA (or typA) gene. Example of this is the GenBank bipA (or typA) annotations in the GenBank references given above.

Below is described some relevant bipA (or typA) gene relevant information, which shall be seen as a mere illustration of common general knowledge with respect to the bipA (or typA) gene.

BipA (or typA), was discovered as a protein strongly upregulated when Salmonella enterica is exposed to the host defense protein BPI (Qi, et al, (1995) Mol. Microbiol. 17, 523-531) Null mutants of BipA (or typA) are pleiotropic, with defects in key processes, including flagella mediated cell motility, growth at low temperatures or low pH, resistance to certain antimicrobial peptides, expression of K5 capsule system, and, at least in the case of enteropathogenic E. coli, BipA (or typA) also rules the expression of two virulence-related gene clusters (Grant, et al, (2003) Mol. Microbiol. 48, 507-521). Bip A (or typA) allows the efficient expression of F is, which is regulated at a transcriptional level, thus modulating a range of downstream processes such as DNA metabolism, and type III secretion (Owens, et al, (2004) Embo J. 23, 3375-3385).

The wide-ranging nature of these processes emphasizes the large-scale regulatory properties exhibited by BipA (or typA). BipA (or typA) binds to ribosomes at a site that coincides with that of elongation factor G, and has a GTPase activity that is sensitive to high GDP:GTP rations, and stimulated by ribosomes programmed with mRNA and aminoacylated tRNAs.

BipA (or typA) is a member of the GTP binding elongation factor family whose main functional characteristic is the regulation of protein biosynthesis. Nevertheless, this protein can be defined in many ways, as it can be found in the literature. These alternative definitions are:

Translation elongation factor
Promoter of GTP-dependent translocation of the nascent protein chain from the A-site to the P-site of the ribosome
Fis regulator at a transcriptional level

With respect to the first aspect of the invention it is for the relevant bipA (or typA) gene said that there shall be at least 95% identity to relevant reference sequences. For the relevant bipA (or typA) gene of the first aspect of the invention the identity percentage is preferably at least 97.5% identity to relevant reference sequences.

Analyzing the Sample to Identify for Presence of a Salmonella biA (or typA) Gene

In step (i) of the first aspect the sample is analyzed to identify for presence of a Salmonella bipA (or typA) gene.

As said above, an advantage of the present invention is that Salmonella can be specifically distinguished from other microorganisms (e.g. other species from the family enterobacteriaceae) present in the sample.

Accordingly, in a preferred embodiment the sample is analyzed by a suitable technique capable of specifically identifying the analyzed Salmonella bipA (or typA) gene from the one or more other microorganism(s) further comprised within the sample.

The art describes a number of such amplification techniques including polymerase chain reaction (PCR) or ligase chain reaction (LCR) based technology. There are also described techniques that may be said to be based on amplification under isothermal conditions, such as NASBA (nucleic acid sequence-based amplification, described in PCT Public. No. WO 91/02818) or the “Strand Displacement Amplification” method, termed SDA, which is described in U.S. Pat. No. 5,270,184.

Use of any of such amplification techniques is a routine task for the skilled person and they represent suitable examples of herein relevant amplification techniques.

Accordingly, a preferred embodiment the method, as described herein, is wherein the analysis, to identify for presence of a Salmonella bipA (or typA) gene in accordance with step (i) of the method, is done by a suitable gene amplification technique [e.g. polymerase chain reaction (PCR), ligase chain reaction (LCR), NASBA (nucleic acid sequence-based amplification) or Strand Displacement Amplification (SDA)] to amplify the relevant gene or mRNA expressed from the gene.

Further it is particular preferred wherein the amplification technique is performed in a way wherein it is capable of specifically amplifying the analyzed Salmonella bipA (or typA) gene and do not amplify measurable amounts of bipA (or typA) gene sequences from the one or more other microorganism(s) further comprised within the sample.

Preferably, the suitable gene amplification technique is PCR (preferably real-time PCR) and wherein the PCR primers are constructed in a way so the PCR primers specifically amplify the analyzed Salmonella bipA (or typA) gene and do not amplify measurable amounts of bipA (or typA) gene sequences from the one or more other microorganism(s) further comprised within the sample.

Preferably there is used real-time PCR combined with suitable fluorescent hybridization techniques to add sensitivity to the detection methods and e.g. considerably shortening the time per analysis.

Further, use of techniques such as PCR in addition allows bacterial load estimation in a given sample by approaching the total number through the quantization of the number of genomic copies of the targeted gene.

In this respect it is a further advantage that there is normally only one copy per genome of the bipA (or typA) Salmonella gene as described herein.

As explained herein, based on common knowledge and the information provided herein it is routine work for the skilled person to make such Salmonella bipA (or typA) gene specific primers and probe. See e.g. working examples herein where it is done for the bipA (or typA) gene.

In working example 1 herein is used the primers and probe shown in SEQ ID NO 5 (termed SAL1410_F), SEQ ID NO 6 (termed SAL 1494_R), and SEQ ID NO 7 (termed SAL1441_PR). Accordingly, in a preferred embodiment the PCR primers are selected from the group of PCR primers consisting of: SEQ ID NO 5 (termed SAL1410_F): 5′-GGT CTG CTG TAC TCC ACC TTC AG-3′; SEQ ID NO 6 (termed SAL 1494_R): 5′ TTG GAG ATC AGT ACG CCG TTC T-3′, and SEQ ID NO 7 (termed SAL1441_PR): 5′-TTA CGA CGA TAT TCG TCC GGG TGA AGT G-3′.

### Identity of DNA Sequences:

The DNA sequence identity referred to herein is determined as the degree of identity between two sequences indicating a deviation of the first sequence from the second.

At the filing date of the present invention, the National Center for Biotechnology Information (NCBI) offered at the Internet site (http://www.ncbi.nlm.nih.gov/) allows the possibility of making a standard BLAST computer sequence homology search.

BLAST program is described in [Altschul et al (1997), “Gapped BLAST and PSI-BLAST: a new generation of protein database search programs”, Nucleic Acids Res. 25:3389-3402].

In the present context, a preferred computer homology search program is a “Standard nucleotide-nucleotide BLAST [blastn]” search as specified, at the filing date of the present application, at the NCBI Internet site with setting filter: Low complexity; Expect: 10, Word Size: 11.

The reference sequence is introduced into the program and the programidentifies fragments of another sequence (e.g. a published sequence) together with the identity percentage to a corresponding fragment of the reference sequence.

According to the common understanding of the skilled person, when there herein is discussed an identity to a specific reference sequence to another sequence, said another sequence should have a length which is comparable to the reference sequence. For instance, if the length of the reference sequence is 200 bp a comparable length of the other sequence could e.g. be from 150-250 bp. The same applies for identity of amino acid sequences as described herein.

### Identity to Amino Acid Sequences

Similar to the nucleotide homology analysis, in the present context, a preferred computer homology search program is a “Standard protein-protein BLAST [blastp]” search as specified, at the filing date of the present application, at the NCBI Internet site with settings Composition-based statistics: yes, filter: Low complexity; Expect: 10, Word Size: 3, Matrix: BLOSUM 62, Gap Costs: Existence 11 Extension 1.

### Examples

### Material and Methods

Organisms Used in this Study

Forty eight serovars of Salmonella were used to test the specificity of the primers and the Taqman probe used. In addition, a total of 30 different bacterial species belonging to all major phylogenetic lineages have been used as negative specificity controls.

### DNA Extraction and Quantitation

DNA from the specimens was extracted by using the kit NucleoSpin Tissue as specified by the manufacturer (Macherei-Nagel). DNA concentration was determined by the PicoGreen™ method (Molecular Probes) by comparing fluorescence values with those of a calibration curve built up from a dilution series of Salmon sperm DNA (Sigma chemicals).

### Primer Design

All available partial sequences of the gene bipA (or typA) in the genbank from Salmonella were obtained and aligned, resulting to be identical. This fragment was then used to design two set of primers. The first one, was intended to be used in conventional PCR assays for those assays requiring a presumptive (presence/absence) determination of Salmonella. Both forward and reverse primers were evaluated with the NetPrimer software (PREMIER Biosoft International, Palo Alto, Calif.) for the formation of primer-dimer structures and hairpins.

The second set of primers and a probe were designed for the determination of Salmonella. by real-time PCR. After introducing the consensus bipA (or typA) sequence in the software Primer Express™ v. 2.0 (Applied Biosystems, Forster City Calif.) optimal primers set SEQ ID NO 5 (5′-GGT CTG CTG TAC TCC ACC TTC AG-3′) (termed SAL1410_F), SEQ ID NO 6 (5′ TTG GAG ATC AGT ACG CCG TTC T-3′) (termed SAL1494_R), and Taqman™ probe SEQ ID NO 7 (5′-TTA CGA CGA TAT TCG TCC GGG TGA AGT G-3′) (termed SAL1441-PR) were obtained.

### PCR Conditions

Conventional PCR was carried out in 25 μl (total volume) reaction mixtures by using a thermal cycler (model 9600 P.E. Applied Biosystems, Foster City, Calif., USA). PCR conditions were 95° C. for 10 min; 40 cycles consisting of 94° C. for 35 sec, 60° C. for 35 sec and 72° C. for 35 sec; and a final extension step consisting of 72° C. for 10 min. Reaction mixtures contained 50-100 ng DNA template, 2.5 mM MgCl₂, 0.25 μM of each primer, 0.8 mM dNTP mix, and 0.5 U of TaqGold (P.E. Applied Biosystems, Forster City, Calif., USA). An internal amplification control consisting of ca. 103 amplicon copies were added to a parallel reaction in order to control false negatives by ensuring that no PCR inhibition was being produced.

Real-time PCR was carried out in 25 μl (total volume) reaction mixtures by using a thermal cycler AbiPrism 7700 and the software SD Sv1.2. PCR conditions were 50° C. for 2 min; 95° C. for 10 min, and 40 cycles consisting of 95° C. for 15 sec, and 60° C. for 1 min. Reaction mixtures were prepared using the Universal Master Mix (P.E. Applied Biosystems, Forster City, Calif. USA), DNA template, and an internal amplification control consisting of ca. 103 amplicon copies which were added into the same reaction in order to control false negatives by ensuring that no PCR inhibition was being produced.

### 2. Results

2.1 The gene bipA (or typA)

The analytical system specifically targets gene bipa (or typA), which unlike the other markers of choice used elsewhere (invA, tyv, prt, viaB) is not unique for Salmonella. It is a widespread gene among bacteria whose functions are not to or dependent on inducible activities such as pathogenesis. Instead, the protein encoded by the gene BipA is essential for sustaining cell life and viability. Both the gene and the protein sequences have been compared with those of related organisms, showing relatively high phylogenetic distances, which considerably eased the task of finding specific oligonucleotides.

2.2 Primer design

A primer set targeting a fragment of around 200 bp of the bipA (or typA) gene was first designed and used in a PCR with genomic DNA of Salmonella. PCR products of the expected size were obtained.

The second primer set and the probe targeting a 84 bp of the bipA (or typA) gene designed were used in a real-time PCR with genomic DNA of Salmonella. The expected signal was observed with the SDS v1.2 software (P.E. Applied Biosystems, Forster City, Calif. USA).

The first primer set was: SAL1504_F. (SEQ ID NO 3) 5′-TTC GGT TTG CAG GAT CG—3′ and SAL1704_R (SEQ ID NO 4) 5′-CGC TTG CTC AAG ACT CAT TTT A-3′. The second primer set and probe were: SAL1410_F., (SEQ ID NO 5) 5′-GGT CTG CTG TAC TCC ACC TTC AG -3′; SAL1494_R, (SEQ ID NO 6) 5′-TTG GAG ATC AGT ACG CCG TTC T -3′ and SAL1441_PR (SEQ ID NO 7) 5′-TTA CGA CGA TAT TCG TCC GGG TGA AGT G -3′.

2.3 Inclusivity-Exclusitivy test

A PCR using genomic DNA of 48 Salmonella serovars and 30 other bacteria from several subgroups of the Proteobacteria as template was performed. Results were positive in all the Salmonella tested (Table 1). Negative results were obtained for the rest of bacterial species representing different taxa and phylogenetic lineages (Table 1).

TABLE 1
Bacteria used in the inclusivity-exclusivity test of the different sets of primers.

| Organism | PCR | IAC |
|---|---|---|
| S. choleraesuis subsp. Arizonae CECT 4395 | + | + |
| S. choleraesuis subsp. Salamae CECT 4000 Type strain | + | + |
| S. choleraesuis subsp. Choleraesuis (S. enteridis var. chaco)CECT 4155 | + | + |
| S. choleraesuis subsp. Choleraesuis (gallinarum) CECT 4182 | + | + |
| S. choleraesuis subsp. Choleraesuis (typhimurium) CECT 4296 | + | + |
| S. choleraesuis subsp. choleraesuis Serovar enteritidis CECT 4371 | + | + |
| S. choleraesuis subsp. choleraesuis Serovar typhi CECT 409 | + | + |
| S. choleraesuis subsp. choleraesuis Serovar paratyhpi CECT 4139 | + | + |
| S. choleraesuis subsp. choleraesuis Serovar urbana CECT 4151 | + | + |
| S. choleraesuis subsp. choleraesuis Serovar dublin CECT 4152 | + | + |
| S. choleraesuis subsp. choleraesuis Serovar saint-paul CECT 4153 | + | + |
| S. choleraesuis subsp. choleraesuis Serovar virchow CECT 4154 | + | + |
| S. typhimurium CECT 4594 | + | + |
| S. typhimurium ECT 443 | + | + |
| Salmonella spp. Isolate 06/162 Toxis LSPG | + | + |
| Salmonella spp. Isolate 05/2069 Toxis LSPG | + | + |
| Salmonella spp. Isolate 05/2070 Toxis LSPG | + | + |
| Salmonella spp. Isolate 05/2071 | + | + |
| Salmonella spp. Isolate 06/299-D1 Toxis LSPG | + | + |
| Salmonella spp. Isolate 04/01 Toxis LSPG | + | + |
| S. enterica, subes I enteritidis lisotip 1 | + | + |
| S. enterica, subes I enteritidis lisotip 1 | + | + |
| S. enterica, subes I enteritidis lisotip 1 | + | + |
| S. enterica, subesp I enteritidis 9.12 lisotip 4 | + | + |
| S. enterica, subesp I enteritidis 9.12 lisotip 1B | + | + |
| S. enterica, serovar Gallinarum | + | + |
| S. enteritidis TE 31154 | + | + |
| S. enteritidis TE 31327 | + | + |
| S. enteritidis TE 31888 | + | + |
| S. enteritidis TE 32271 | + | + |
| S. enteritidis TE 32302 | + | + |
| S. enteritidis TE 32337 | + | + |
| S. enteritidis TE 32395 | + | + |
| S. enteritidis TE 64752 | + | + |
| S. enteritidis TE 75108 | + | + |
| S. enteritidis TE 85230 | + | + |
| S. enteritidis TE 232 | + | + |
| S. typhimurium TT 30018 | + | + |
| S. typhimurium TT 31980 | + | + |
| S. typhimurium TT 31658 | + | + |
| S. typhimurium TT 32050 | + | + |
| S. typhimurium TT54336 | + | + |
| S. typhimurium TT 55402 | + | + |
| S. typhimurium TT 64472 | + | + |
| S. typhimurium TT 67090 | + | + |
| S. typhimurium TT 88301 | + | + |
| S. typhimurium TT 98881 | + | + |
| Salmonella LT2 | + | + |
| Shigella spp | − | + |
| Pseudomonas fluorescens | − | + |
| Arthrobacter VPI | − | + |
| Shigella sonnei | − | + |
| Serratia marcescens | − | + |
| Pseudomonas aeruginosa | − | + |
| Enterobacter aerogenes | − | + |
| Proteus mirabilis | − | + |
| Micrococcus luteus | − | + |
| Bacillus subtilis | − | + |
| Bacillus megaterium | − | + |
| Bacillus cereus | − | + |
| Staphylococcus epidermidis | − | + |
| Enterococcus faecalis | − | + |
| Enterobacter cloacae | − | + |
| Staphylococcus aureus | − | + |
| Citrobacter | − | + |
| Shigella sonnei CECT 4631 | − | + |
| E. coli CECT 434 | − | + |
| Enterococcus faecalis CECT 795 | − | + |
| Staphylococcus aureus CECT 435 | − | + |
| Pseudomonas aeruginosa CECT 108 | − | + |
| Clostridium perfringens CECT 376 | − | + |
| Enterobacter cloacae CECT 194 | − | + |
| Listeria inocua CECT 910 | − | + |
| Listeria monocytogenes CECT 4032 | − | + |
| Bacillus cereus CECT 148 | − | + |
| E. coli O157:H7 CECT 4267 | − | + |
| Yersinia enterocolitica biovar CECT 4315 | − | + |
| Legionella pneumophila ATCC 33152 | − | + |

IAC stands for Internal Amplification Control

1. A method for specific detection of the presence of Salmonella spp. in a sample that is suspected to contain Salmonella spp. and which contains one or more other microorganism(s); the method comprising:
(i) analyzing the sample to identify for presence of a Salmonella bipA (or typA) gene; and
(ii) evaluating the amount of the Salmonella bipA (or typA) gene present in the sample and if the sample contains the Salmonella bipA (or typA) gene it is proof that Salmonella is present in the sample;
wherein the Salmonella bipA (or typA) gene is a bipA (or typA) gene selected from the group of bipA (or typA) genes consisting of:
(a) a bipA (or typA) gene having a DNA sequence which is at least 95% identical to the DNA sequence shown in positions 1-1824 of SEQ ID NO 1 (termed “bipA (or typA)”); and
(a1) a bipA (or typA) gene that encodes a polypeptide which is at least 95% identical to the polypeptide shown in positions 1-607 of SEQ ID NO 2 (termed BipA (or TypA) GTPase).

2. The method of claim 1, wherein the one or more other microorganism(s) of species other than Salmonella spp. in the sample are one or more of E. coli, Shigella, Enterobacter, Micrococcus, Bacillus, Staphylococcus, Pseudomonas, Serratia, Proteus, Enterococcus, Arthrobacter and Listeria.

3. The method of claim 1, wherein the sample is an alimentary sample.

4. The method of claim 1, wherein the Salmonella bipA (or typA) gene is a bipA (or typA) gene selected from a group of bipA (or typA) genes consisting of:
(a) a bipA (or typA) gene having-a DNA sequence which is identical to the DNA sequence shown in positions 1-1824 SEQ ID NO 1 (termed bipA (or typA)); and
(a1) a bipA (or typA) gene that encodes a polypeptide which is identical the polypeptide shown in positions 1-607 SEQ ID NO 2 (termed BipA (or TypA) GTPase).

5. The method of claim 1, wherein the analysis to identify for the presence of a Salmonella bipA (or typA) gene or with step (i) of the method, is done by a gene amplification technique to amplify the relevant gene and wherein the amplification technique is capable of specifically amplifying the analyzed Salmonella bipa (or typA) gene and does not amplify measurable amounts of bipa (or typA) gene sequences from the one or more other microorganism(s) further comprised within the sample.

6. The method of claim 5, wherein the suitable gene amplification technique is PCR and wherein the PCR primers are constructed in a way so the PCR primers specifically amplify the analyzed Salmonella bipea (or typA) gene and do not amplify measurable amounts of bipa (or typA) gene sequences from the one or more other microorganism(s) further comprised within the sample.

7. The method of claim 6, wherein the PCR primers are at least one primer selected from the group of PCR primers consisting of:
SEQ ID NO 3 (termed SAL1504_F): 5′-TTC GGT TTG CAG GAT CG -3′;
SEQ ID NO 4 (termed SAL1704_R): 5′-CGC TTG CTC AAG ACT CAT TTT A-3′;
SEQ ID NO 5 (termed SAL1410_F): 5′-GGT CTG CTG TAC TCC ACC TTC AG -3′;
SEQ ID NO 6 (termed SAL1494_R): 5′-TTG GAG ATC AGT ACG CCG TTC T -3′: and
SEQ ID NO 7 (termed SAL1441_PR): 5′-TTA CGA CGA TAT TCG TCC GGG TGA AGT G -3′.

8. The method of claim 3, wherein the alimentary sample is selected from the group consisting of egg, poultry, raw (under cooked) meat, raw seafood, milk, and dairy products, water, sauces and salad dressings.

9. The method of claim 5, wherein the gene amplification technique is selected from the group consisting of polymerase chain reaction (PCR), ligase chain reaction (LCR), NASBA (nucleic acid sequence-based amplification) and Strand Displacement Amplification (SDA).

10. The method of claim 6, wherein the suitable gene amplification technique is real-time PCR.


SEQUENCE LISTING (7 sequences)

SEQ ID NO: 1 - DNA, 1824 bp, Salmonella enterica
  CDS (1)..(1824)
atgatcgaaaatttgcgtaacatcgccatcatcgcgcacgttgaccatggtaaaactaccctggttgataagctgctccagcaatccggtacgttcgacgcgcgtgccgaaactcaagagcgagtgatggactccaacgatttggagaaagagcgtggtattactatcctcgctaaaaacaccgctattaaatggaatgattaccgtatcaacatcgttgatactcccgggcacgctgacttcggtggtgaagtagagcgcgtcatgtccatggttgactctgtgctgctggtggttgacgcatttgacggcccgatgccgcaaacgcgcttcgtgaccaaaaaagcctttgctcatggcctgaaacccattgtggttatcaacaaagttgaccgtcctggcgcgcgccctgactgggttgttgaccaggtattcgacctgtttgttaacctcgacgcgaccgacgaacagctggacttcccgatcatctacgcttcggcgctgaacggtatcgcgggtctggaccacgaagatatggcggaagacatgaccccgctatatcaggcgattgttgatcatgttccggcgccggacgttgacctcgatggtccgttgcagatgcagatctcccagctggactacaacaactatgttggcgttatcggcattggccgtatcaaacgcggcaaagtgaagccgaaccagcaggtcactatcatcgatagtgaagggaaaacccgtaacgcgaaagtaggtaaagtgctgacgcatctgggtctggagcgtatcgacagcaatatcgccgaagcgggcgatatcattgcgatcaccggtctgggcgagctgaacatttccgacaccatctgcgacccgcagaacgttgaagcgctgccggcgctgtccgttgatgagccgaccgtgtctatgttcttctgcgttaacacctcgccgttctgcggtaaagaaggtaagtttgtgacttctcgtcagattcttgaccgtctgaacaaagagctggtgcataacgtggcgctgcgcgttgaagaaaccgaagatgcggatgcgttccgtgtatccggtcgtggcgaactgcacctgtccgtgctgattgaaaatatgcgtcgtgaaggtttcgaactggcggtttcccgtccgaaagttatcttccgtgaaatcgacggtcgtaaacaagagccgtacgaaaacgtgacgctggacgtcgaagagcagcaccaggggtctgtcatgcaggcgctgggcgagcgtaaaggcgacctgaaaaacatgaatccggacggtaaaggccgcgtacgtctcgactacgtgatcccaagccgtgggctgattggtttccgttcagaattcatgaccatgacttccggtacgggtctgctgtactccaccttcagccattacgacgatattcgtccgggtgaagtgggccagcgtcagaacggcgtactgatctccaacggtcagggtaaagcggtggcgtttgcgctgttcggtttgcaggatcgcggtaagctgttcctgggtcacggcgcggaagtttatgaaggccagattattggtattcacagtcgctccaacgacctgacggtaaactgcctgaccggtaagaaactgaccaacatgcgtgcgtccggtacggatgaagcggtgattctggttccgccaattaaaatgagccttgagcaagcgctggagttcattgatgacgacgaactggtagaagtcaccccaacctctatccgtatccgtaaacgtcacctgacggaaaacgatcgccgccgtgcgaaccgtggtcagaaagaagagtaa

SEQ ID NO: 2 - protein, 607 aa, Salmonella enterica
MIENLRNIAIIAHVDHGKTTLVDKLLQQSGTFDARAETQERVMDSNDLEKERGITILAKNTAIKWNDYRINIVDTPGHADFGGEVERVMSMVDSVLLVVDAFDGPMPQTRFVTKKAFAHGLKPIVVINKVDRPGARPDWVVDQVFDLFVNLDATDEQLDFPIIYASALNGIAGLDHEDMAEDMTPLYQAIVDHVPAPDVDLDGPLQMQISQLDYNNYVGVIGIGRIKRGKVKPNQQVTIIDSEGKTRNAKVGKVLTHLGLERIDSNIAEAGDIIAITGLGELNISDTICDPQNVEALPALSVDEPTVSMFFCVNTSPFCGKEGKFVTSRQILDRLNKELVHNVALRVEETEDADAFRVSGRGELHLSVLIENMRREGFELAVSRPKVIFREIDGRKQEPYENVTLDVEEQHQGSVMQALGERKGDLKNMNPDGKGRVRLDYVIPSRGLIGFRSEFMTMTSGTGLLYSTFSHYDDIRPGEVGQRQNGVLISNGQGKAVAFALFGLQDRGKLFLGHGAEVYEGQIIGIHSRSNDLTVNCLTGKKLTNMRASGTDEAVILVPPIKMSLEQALEFIDDDELVEVTPTSIRIRKRHLTENDRRRANRGQKEE

SEQ ID NO: 3 - DNA, 17 bp, Artificial
  PCR primer
ttcggtttgcaggatcg

SEQ ID NO: 4 - DNA, 22 bp, Artificial
  PCR primer
cgcttgctcaagactcatttta

SEQ ID NO: 5 - DNA, 23 bp, Artificial
  PCR primer
ggtctgctgtactccaccttcag

SEQ ID NO: 6 - DNA, 22 bp, Artificial
  PCR primer
ttggagatcagtacgccgttct

SEQ ID NO: 7 - DNA, 28 bp, Artificial
  PCR primer
ttacgacgatattcgtccgggtgaagtg